(19) United States
(12) Patent Application Publication
(10) Pub. No.: US 2016/0206836 A1
(43) Pub. Date: Jul. 21, 2016

(54) SYSTEM AND METHOD FOR AIDING BREATHING

(71) Applicant: HTC Corporation, Taoyuan City (TW)

(72) Inventors: Yen-Liang KUO, Taoyuan City (TW); Chun-Yih WU, Taoyuan City (TW); Chih-Jen HU, Taoyuan City (TW)

(21) Appl. No.: 14/598,198

(22) Filed: Jan. 15, 2015

(51) Int. Cl. A61M 16/00; A61M 16/20; A61M 16/06

(52) U.S. Cl. CPC A61M 16/0069; A61M 16/0003; A61M 16/06; A61M 16/201; A61M 2016/0015; A61M 2205/3334; A61M 2205/3546; A61M 2205/60; A61M 2230/63

(57) ABSTRACT

A system for aiding breathing is provided. The system includes a breathing detection device, a sound-receiving device, an airflow providing device and a processing device. The breathing detection device detects a body displacement of a target, wherein the body displacement corresponds to a breathing of the target. The sound-receiving device detects a breathing sound of the target. The airflow providing device generates an airflow. The processing device determines a breathing condition of the target according to the body displacement and further determines whether a sleep apnea condition of the target occurs according to the breathing condition and the breathing sound. The processing device controls the airflow providing device to generate the airflow to a respiratory organ of the target when the sleep apnea condition occurs.

### BACKGROUND

1. Field of Invention

The present invention relates to a technology for aiding breathing. More particularly, the present invention relates to a system for aiding breathing and a method of the same.

2. Description of Related Art

Obstructive sleep apnea is a disease that a breathing of a patient becomes too slow or even stops during sleeping due to the obstruction of respiratory tract nasal caused by the obstruction of the tissue of the cavity and the throat. The obstruction may last for a few seconds to dozens of seconds. Further, snoring of the patient becomes irregular such that the frequency of snoring of the patient is unstable. The stop of the breathing makes the patient wake up from sleeping frequently and results in insufficient sleep. The patient therefore feels tired easily during daytime and may not work efficiently.

Continuous positive airway pressure (CPAP) device is often used as the treatment for the patient having the obstructive sleep apnea. In order to avoid the airflow of breathing being obstructed, the CPAP device keeps providing high pressure oxygen through a mask even the symptom does not occur. However, the continuous airflow would make the patient uncomfortable. The sleeping quality is still not improved well.

Accordingly, what is needed is a system for aiding breathing and a method of the same to address the issues mentioned above.

### SUMMARY

An aspect of the present invention is to provide a system for aiding breathing. The system includes a breathing detection device, a sound-receiving device, an airflow providing device and a processing device. The breathing detection device detects a body displacement of a target, wherein the body displacement corresponds to a breathing of the target. The sound-receiving device detects a breathing sound of the target. The airflow providing device generates an airflow. The processing device determines a breathing condition of the target according to the body displacement and further determines whether a sleep apnea condition of the target occurs according to the breathing condition and the breathing sound, such that when the sleep apnea condition occurs, the processing device controls the airflow providing device to generate the airflow to a respiratory organ of the target.

According to an embodiment of the present invention, the breathing detection device is a radar device to perform a transmission and reception of a wireless wave on the target to detect the body displacement.

According to another embodiment of the present invention, the processing device stores an algorithm therein to perform calculation according to the breathing condition and the breathing sound, such that when a stop time of the breathing of the target exceeds a certain time and the breathing sound reveals a frequency change of the snoring behavior, the processing device determines that the sleep apnea condition of the target occurs.

According to yet another embodiment of the present invention, the airflow providing device includes an air supplying mask and an air supplying device, wherein the air supplying mask is disposed on a mouth and a nose of the target.

According to further another embodiment of the present invention, the air supplying device is a high pressure gas cylinder, and the airflow providing device further includes a valve such that the processing device opens the valve to allow the target breathing when the sleep apnea condition does not occur, and closes the valve to control the high pressure gas cylinder to transmit the airflow to the mouth and the nose of the target through the air supplying mask when the sleep apnea condition occurs.

Another aspect of the present invention is to provide a method for aiding breathing used in a system for aiding breathing. The method includes the steps outlined below. A body displacement of a target is detected by a breathing detection device of the system for aiding breathing, wherein the body displacement corresponds to a breathing of the target. A breathing sound of the target is detected by a sound-receiving device of the system for aiding breathing. A breathing condition of the target is determined by a processing device of the system for aiding breathing according to the body displacement. Whether a sleep apnea condition of the target occurs is determined by the processing device according to the breathing condition and the breathing sound. An airflow providing device of the system for aiding breathing is controlled by the processing device to generate the airflow to a respiratory organ of the target when the sleep apnea condition occurs.

According to an embodiment of the present invention, the breathing detection device is a radar device, and the step of detecting the body displacement further includes the steps outlined below. A transmission and reception of a wireless wave on the target is performed by the radar device. The body displacement is detected according to the transmission and reception of the wireless wave.

According to another embodiment of the present invention, the processing device stores an algorithm therein, and the step of determining whether the sleep apnea condition of the target occurs further includes the steps outlined below. Calculation is performed according to the breathing condition and the breathing sound by the processing device. The processing device determines that the sleep apnea condition of the target occurs when a stop time of the breathing of the target exceeds a certain time and the breathing sound reveals a frequency change of the snoring behavior.

According to yet another embodiment of the present invention, the airflow providing device includes an air supplying mask and an air supplying device, wherein the air supplying mask is disposed on a mouth and a nose of the target.

According to further another embodiment of the present invention, the air supplying device is a high pressure gas cylinder connected to the air supplying mask through a valve, the method further includes the steps outlined below. The valve is opened by the processing device to allow the target breathing when the sleep apnea condition of the target does not occur. The valve is dosed by the processing device to control the high pressure gas cylinder to transmit the airflow to the mouth and the nose of the target through the air supplying mask when the sleep apnea condition of the target occurs.

These and other features, aspects, and advantages of the present invention will become better understood with reference to the following description and appended claims.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a block diagram of a system for aiding breathing and a target in an embodiment of the present invention;

FIG. 2 is a diagram of the components of the system for aiding breathing and the target in an embodiment of the present invention;

FIG. 3 is a flow chart of a method for aiding breathing in an embodiment of the present invention;

FIG. 4 is a diagram of the components of the system for aiding breathing and the target in an embodiment of the present invention; and

FIG. 5 is a flow chart of a method for aiding breathing in an embodiment of the present invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

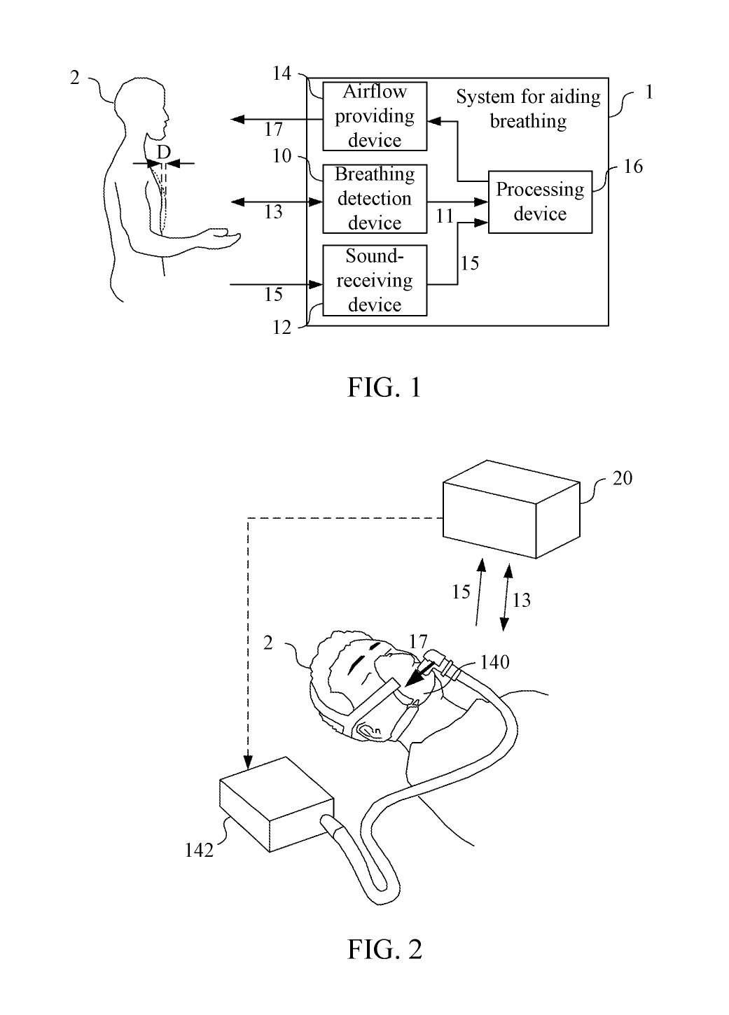

FIG. 1 is a block diagram of a system 1 for aiding breathing and a target 2 in an embodiment of the present invention. The system 1 includes a breathing detection device 10, a sound-receiving device 12, an airflow providing device 14 and a processing device 16.

The breathing detection device 10 detects a body displacement 11 of a target 2, wherein the body displacement 11 corresponds to a breathing of the target 2. Since when the target 2 breaths, a displacement is generated at some positions of the body, such as but not limited to chest, stomach or other positions. Therefore, the body displacement 11 may correspond to the displacement D of one of the positions of the body mentioned above.

In an embodiment, the breathing detection device 10 is a radar device to perform a transmission and reception of a wireless wave 13 on the positions of the target 2 to detect the body displacement 11. In different embodiments, the wireless wave 13 can be such as, but not limited to a supersonic wave, an infrared wave or a wireless signal in other formats. By using the time difference between the transmission and the reception of the wireless wave 13, the breathing detection device 10 detects the body displacement 11 of the target 2. The processing device 16 determines that whether the target 2 is within an inhaling stage or a exhaling stage accordingly to further determines the breathing condition of the target 2. In an embodiment, the breathing condition includes such as, but not limited to the frequency of the breathing, the depth of the breathing or a combination of the above.

The sound-receiving device 12 detects a breathing sound 15 of the target 2. The processing device 16 determines whether the breathing sound 15 reveals a frequency change of the snoring behavior.

In an embodiment, the processing device 16 stores an algorithm therein to perform calculation according to the breathing condition and the breathing sound 15 determined based on the body displacement 11. When the calculation result reveals that a stop time of the breathing of the target 2 exceeds a certain time and the breathing sound 15 reveals a frequency change of the snoring behavior, the processing device 16 determines that the sleep apnea condition of the target 2 occurs.

For example, after the processing device 16 performs the stored algorithm, the calculation result reveals that the stop time of the breathing of the target 2 exceeds five seconds and the breathing sound 15 reveals that the target 2 starts to snore or the frequency of the snoring behavior increases, the processing device 16 determines that the sleep apnea condition of the target 2 occurs. In an embodiment, the sleep apnea condition can be such as, but not limited to the obstruction of respiratory tract nasal caused by the obstruction of the tissue of the cavity and the throat.

After the processing device 16 determines that the sleep apnea condition occurs, the processing device 16 controls the airflow providing device 14 to generate the airflow 17 to a respiratory organ of the target 2, such as but not limited to the mouth and the nose.

Reference is now made to FIG. 2 together with FIG. 1. FIG. 2 is a diagram of the components of the system 1 for aiding breathing and the target 2 in an embodiment of the present invention.

In an embodiment, the breathing detection device 10, the sound-receiving device 12 and the processing device 16 are integrated in a composite device 20. The composite device 20 is disposed in front of such as, but not limited to the chest and the stomach of the target 2. Accordingly, the breathing detection device 10 performs the transmission and the reception of the wireless wave 13 by using the mechanism mentioned above to detect the body displacement 11 of the target 2 and further determines the breathing condition of the target 2. The sound-receiving device 12 detects the breathing sound 15 of the target 2. The processing device 16 further determines whether the sleep apnea condition of the target 2 occurs according to the detection of the breathing detection device 10 and the sound-receiving device 12.

In an embodiment, the airflow providing device 14 illustrated in FIG.1 includes the air supplying mask 140 and the air supplying device 142, in which the air supplying mask 140 is disposed on the mouth and the nose of the target 2. In an embodiment, the air supplying mask 140 and the air supplying device 142 are connected through a wire. When the sleep apnea condition occurs, the processing device 16 controls the air supplying device 142 of the airflow providing device 14 to generate the airflow 17. The airflow 17 is transmitted to the mouth and the nose of the target 2 through the air supplying mask 140 due to the high pressure of the airflow 17 to make of respiratory tract of the target 2 clear.

In different embodiments, the processing device 16 of the composite device 20 performs communication with and controls the air supplying device 142 of the airflow providing device 14 through various kinds of wired or wireless method.

It is noted that the physical structure described above is merely an example. In other embodiments, the disposition of the components described above can be adjusted according to practical conditions and is not limited thereto.

Consequently, the system 1 for aiding breathing of the present invention can detect the breathing of the target 2 instantly by using the breathing detection device 10 and the sound-receiving device 12 to determine whether the sleep apnea condition of the target occurs. Only when the sleep apnea condition of the target occurs, the airflow providing device 14 is controlled to generate the airflow 17 to the target 2. The uneasiness of the target 2 caused by the continuous airflow 17 supplied to the user can be avoided.

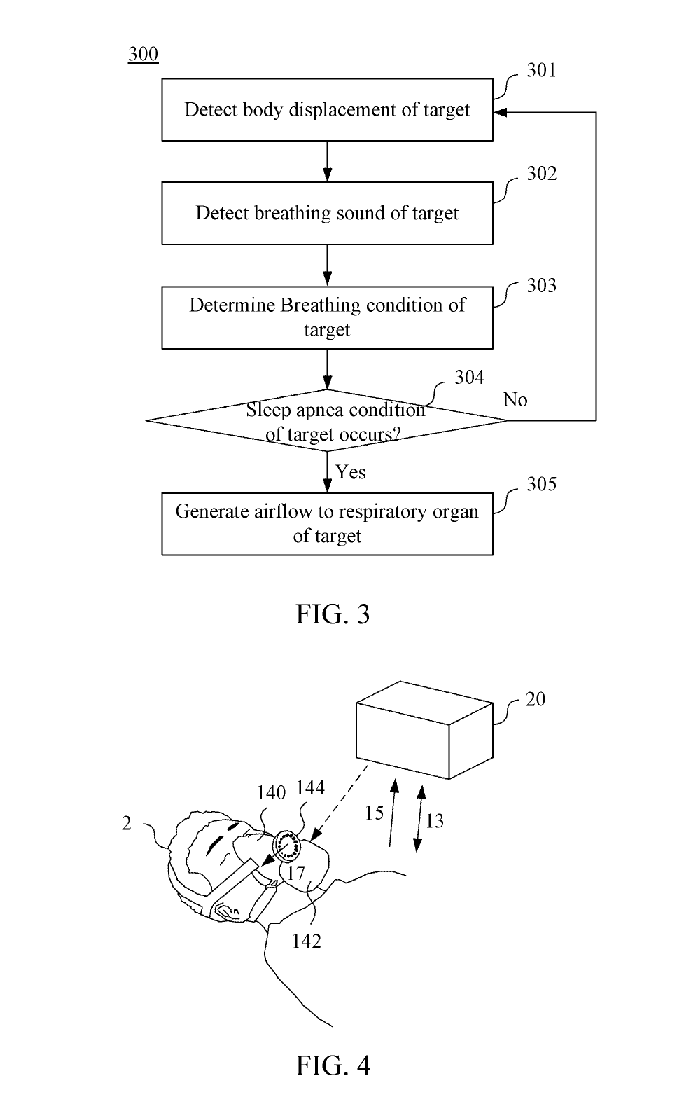

FIG. 3 is a flow chart of a method 300 for aiding breathing in an embodiment of the present invention. The method 300 is used in the system 1 for aiding breathing illustrated in FIG. 1 and FIG. 2. The method 300 includes the steps outlined below (The steps are not recited in the sequence in which the steps are performed. That is, unless the sequence of the steps is expressly indicated, the sequence of the steps is interchangeable, and all or part of the steps may be simultaneously, partially simultaneously, or sequentially performed).

In step 301, the body displacement 11 of the target 2 is detected by a breathing detection device 10 of the system 2 for aiding breathing, wherein the body displacement 11 corresponds to the breathing of the target 2.

In step 302, the breathing sound 15 of the target 2 is detected by the sound-receiving device 12 of the system 1 for aiding breathing.

In step 303, the breathing condition of the target 2 is determined by the processing device 16 of the system 1 for aiding breathing according to the body displacement 11.

In step 304, whether the sleep apnea condition of the target 2 occurs is determined by the processing device 16 according to the breathing condition and the breathing sound 15 based on the stored algorithm.

When the sleep apnea condition of the target 2 does not occur, the flow goes back to the step 301 to keep detecting.

When the sleep apnea condition occurs, the processing device 16 control the airflow providing device 14 of the system 1 for aiding breathing to generate the airflow 17 to the respiratory organ of the target 2 in step 305.

Reference is now made to FIG. 4 together with FIG. 1. FIG. 4 is a diagram of the components of the system 1 for aiding breathing and the target 2 in an embodiment of the present invention.

As described in the previous embodiment, the breathing detection device 10, the sound-receiving device 12 and the processing device 16 are integrated in a composite device 20. As a result, the detail is not further described herein.

In the present embodiment, the airflow providing device 14 illustrated in FIG. 1 includes the air supplying mask 140, the air supplying device 142 and a valve 144, as illustrated in FIG. 4. The air supplying mask 140 is disposed on the mouth and the nose of the target 2. The air supplying device 142 is a high pressure gas cylinder.

In the present embodiment, the processing device 16 opens the valve 144 to allow the target 2 breathing when the sleep apnea condition does not occur. Further, the processing device 16 closes the valve 144 to control the high pressure gas cylinder to transmit the airflow 17 to the mouth and the nose of the target 2 through the air supplying mask 140 when the sleep apnea condition occurs.

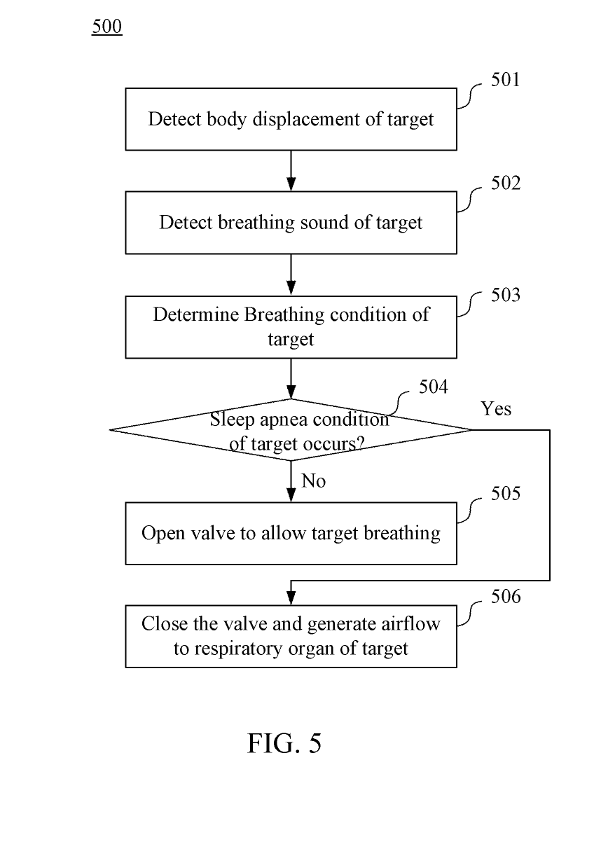

FIG. 5 is a flow chart of a method 500 for aiding breathing in an embodiment of the present invention. The method 500 is used in the system 1 and 4 for aiding breathing illustrated in FIG. 1 and FIG. 4. The method 500 includes the steps outlined below (The steps are not recited in the sequence in which the steps are performed. That is, unless the sequence of the steps is expressly indicated, the sequence of the steps is interchangeable, and all or part of the steps may be simultaneously, partially simultaneously, or sequentially performed).

In step 501, the body displacement 11 of the target 2 is detected by a breathing detection device 10 of the system 2 for aiding breathing, wherein the body displacement 11 corresponds to the breathing of the target 2.

In step 502, the breathing sound 15 of the target 2 is detected by the sound-receiving device 12 of the system 1 for aiding breathing.

In step 503, the breathing condition of the target 2 is determined by the processing device 16 of the system 1 for aiding breathing according to the body displacement 11.

In step 504, whether the sleep apnea condition of the target 2 occurs is determined by the processing device 16 according to the breathing condition and the breathing sound 15 based on the stored algorithm.

When the sleep apnea condition of the target 2 does not occur, the flow goes to the step 505 to open the valve 144 to allow the user breathing.

When the sleep apnea condition occurs, the processing device 16 close the valve 144 to control the airflow providing device 14 of the system 1 for aiding breathing to generate the airflow 17 to the respiratory organ of the target 2 in step 506.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A system for aiding breathing, comprising:
a breathing detection device to detect a body displacement of a target, wherein the body displacement corresponds to a breathing of the target;
a sound-receiving device to detect a breathing sound of the target;
an airflow providing device to generate an airflow; and
a processing device to determine a breathing condition of the target according to the body displacement and further determine whether a sleep apnea condition of the target occurs according to the breathing condition and the breathing sound, such that when the sleep apnea condition occurs, the processing device controls the airflow providing device to generate the airflow to a respiratory organ of the target.

2. The system of claim 1, wherein the breathing detection device is a radar device to perform a transmission and reception of a wireless wave on the target to detect the body displacement.

3. The system of claim 1, wherein the processing device stores an algorithm therein to perform calculation according to the breathing condition and the breathing sound, such that when a stop time of the breathing of the target exceeds a certain time and the breathing sound reveals a frequency change of the snoring behavior, the processing device determines that the sleep apnea condition of the target occurs.

4. The system of claim 1, wherein the airflow providing device comprises an air supplying mask and an air supplying device, wherein the air supplying mask is disposed on a mouth and a nose of the target.

5. The system of claim 4, wherein the air supplying device is a high pressure gas cylinder, and the airflow providing device further comprises a valve such that the processing device opens the valve to allow the target breathing when the sleep apnea condition does not occur, and closes the valve to control the high pressure gas cylinder to transmit the airflow to the mouth and the nose of the target through the air supplying mask when the sleep apnea condition occurs.

6. A method for aiding breathing used in a system for aiding breathing, wherein the method comprises:
detecting a body displacement of a target by a breathing detection device of the system for aiding breathing, wherein the body displacement corresponds to a breathing of the target;
detecting a breathing sound of the target by a sound-receiving device of the system for aiding breathing;
determining a breathing condition of the target by a processing device of the system for aiding breathing according to the body displacement;
determining whether a sleep apnea condition of the target occurs by the processing device according to the breathing condition and the breathing sound; and
controlling an airflow providing device of the system for aiding breathing by the processing device to generate the airflow to a respiratory organ of the target when the sleep apnea condition occurs.

7. The method of claim 6, wherein the breathing detection device is a radar device, and the step of detecting the body displacement further comprises:
performing a transmission and reception of a wireless wave on the target by the radar device; and
detecting the body displacement according to the transmission and reception of the wireless wave.

8. The method of claim 6, wherein the processing device stores an algorithm therein, and the step of determining whether the sleep apnea condition of the target occurs further comprises:
performing calculation according to the breathing condition and the breathing sound by the processing device; and
determining that the sleep apnea condition of the target occurs by the processing device when a stop time of the breathing of the target exceeds a certain time and the breathing sound reveals a frequency change of the snoring behavior.

9. The method of claim 6, wherein the airflow providing device comprises an air supplying mask and an air supplying device, wherein the air supplying mask is disposed on a mouth and a nose of the target.

10. The method of claim 9, wherein the air supplying device is a high pressure gas cylinder connected to the air supplying mask through a valve, the method further comprises:
opening the valve by the processing device to allow the target breathing when the sleep apnea condition of the target does not occur; and
dosing the valve by the processing device to control the high pressure gas cylinder to transmit the airflow to the mouth and the nose of the target through the air supplying mask when the sleep apnea condition of the target occurs.
